(19) United States
(12) Patent Application Publication
(10) Pub. No.: US 2006/0068005 A1
(43) Pub. Date: Mar. 30, 2006

(54) Chewable electrolyte tablet

(75) Inventors: Neil Ross, Miami, FL (US); Jerome Reyman, Miami, FL (US)

Correspondence Address: AKERMAN SENTERFITT, P.O. BOX 3188, WEST PALM BEACH, FL, 33402-3188, US

(73) Assignee: PAL Laboratories, Inc., Miami, FL (US)

(21) Appl. No.: 10/954,874

(22) Filed: Sep. 30, 2004

(51) Int. Cl. A61K 9/20; A61K 31/4188; A61K 31/4172; A61K 31/405; A61K 31/198

(52) U.S. Cl. 424/464; 514/561; 514/393; 514/400; 514/419; 514/562

(57) ABSTRACT

A chewable electrolyte tablet. The chewable electrolyte tablet includes an agent to prevent stomach irritation, a flavoring agent, a sweetening agent, or a combination thereof to enable the tablet to be palatable and, therefore, ingested through simple mastication and without the need for water, thereby permitting the amount of water to be regulated. The tablet may be used to deliver electrolytes, amino acids and/or vitamins. Select embodiments of the tablet contain substantially no carbohydrates, thereby reducing the caloric content of the tablet. In other embodiments, the tablet provides a sustained release of nutrients over time.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

The invention relates to nutritional supplements. More specifically, the invention relates to electrolyte compositions.

### BACKGROUND OF THE INVENTION

Nutritional bars and energy drinks are convenient nutritional supplements, particularly for those persons too busy to eat regular meals and for hikers, cyclists, runners or other athletes who need prepackaged, ready-to-eat, high-energy snacks while they are exercising. Such bars and drinks are also convenient nutritional supplements for the elderly who need prepackaged, ready-to-eat snacks. Additionally, such food supplements may supply consumers with the necessary vitamins and minerals specified in the recommended daily allowances provided by the U.S. government. Nevertheless, many of these nutritional supplement bars are difficult to eat during activity, have an unpalatable taste, and/or provide excess calories that could defeat one of the benefits of exercise.

The use of sport drinks to satisfy various requirements of athletes during training and competition is known. Known drinks are used to provide energy and replace lost electrolytes and nutrients. Sport drinks that are currently on the market generally fail on different levels to provide adequate replenishment. Some drinks contain only sugar and water. As such, these drinks provide no nutritional value and high levels of carbohydrates. Others drinks provide some electrolytes, but again are of little nutritional value and include elevated levels of carbohydrates. Still other drinks contain sugars, some electrolytes, and some vitamins, but also fail to provide much nutritional value and include elevated levels of carbohydrates. Yet other drinks include sugars, some electrolytes, some vitamins, and some minerals. However, as with all other sports drinks, include elevated levels of carbohydrates and do not provide adequate nutritional benefit.

None of the prior art drinks may be used as the exclusive source of electrolyte support during periods of training or competition, as none of the available sports drinks will provide for substantially all of the nutritional requirements of the body under stress. Another disadvantage of these drinks is that they have low gastrointestinal tolerance. As a result, the drinks may cause distention of the bowel, thus creating pressure, pain and cramps from increased peristalsis.

Other prior art nutritional supplements are generally not optimized to provide maximal rates of absorption. Foods that are slowly absorbed during digestion do not provide the body with immediate energy, whereas nutrients which quickly cross the mucosal membranes into the blood provide more immediate energy. Moreover, sports drinks and foods that require large amounts of water will not be optimally beneficial to the athlete during training and competition as the large amounts of water may create an increasing fluid volume in the large bowel, causing diarrhea. Diarrhea causes dehydration and magnifies the electrolyte imbalance that has a catastrophic impact on athletes undergoing the stress of training and competition. On the other hand, as a result of sustained exertion and physical stress, digestion of complex materials may proceed too slowly to provide adequate nutrition to the athlete during such exertion.

An additional disadvantage of the presently available sports drinks is that few have a palatable taste. Taste or palatability is an important parameter for a sports drink or electrolyte support source. Excellent taste is not easily achieved for a sports drink or electrolyte support source for an athlete as the sense of taste changes with the metabolic state of the individual.

Accordingly, it would be beneficial to provide a nutritional supplement having better taste than prior art supplements. It would also be beneficial to provide an electrolyte composition that may be taken without water or any other consumable liquid. It would also be beneficial to provide an electrolyte composition having reduced levels of carbohydrates. It would also be beneficial to provide an electrolyte composition that does not irritate the gastrointestinal system of the person taking the supplement.

### SUMMARY OF THE INVENTION

The present invention relates to a chewable electrolyte tablet. The chewable electrolyte tablet may be designed to be ingested through simple mastication and/or without the need for water, thereby permitting the user to control the amount of water taken with the tablet. This may be accomplished through the use of an agent to prevent stomach irritation, a flavoring agent, a sweetening agent, or a combination thereof. The sweetening agent may be a carbohydrate-free sweetening agent, thereby reducing the caloric content of the tablet. The tablet may be used to deliver electrolytes, amino acids and/or vitamins. In other embodiments, the chewable electrolyte tablet is designed to provide a sustained release of electrolytes, amino acids and/or vitamins over time.

In particular, in one aspect, the present invention provides an electrolyte tablet including at least one nutritional compound and a non-carbohydrate sweetening agent, wherein the electrolyte is chewable such that it may be ingested with little or no water.

In another aspect, the present invention provides sustained-release electrolyte tablet including at least one sustained-release nutritional compound, wherein the at least one sustained-release nutritional compound is released over a period of time of from about 2 hours to about 24 hours. In an alternative embodiment, the sustained-release nutritional compound is released over a period of time of from about 4 hours to about 12 hours.

In yet another aspect, the present invention provides a chewable electrolyte tablet including a composition having the following formulation:

| INGREDIENT | DOSE RANGE POSSIBLE |
|---|---|
| Magnesium | (5 mg to 200 mg) |
| Zinc | (1 mg to 40 mg) |
| Chromium | (.005 to .200 mg) |
| Sodium | (1 mg to 700 mg) |
| Chloride | (1 mg to 700 mg) |
| Potassium | (1 mg to 700 mg) |
| Calcium | (1 mg to 1000 mg) |
| Ascorbic acid | (1 mg to 700 mg) |
| Pyridoxine (HCl) | (.1 mg to 100 mg) |
| Non-Carbohydrate sweetening agent | (100 mg to 3000 mg) |
| Flavoring agent | (1 mg to 400 mg) |
| Gastrointestinal soothing agent | (1 mg to 300 mg) |

wherein the electrolyte is chewable such that it may be ingested with little or no water.

### DETAILED DESCRIPTION OF THE INVENTION

The present invention is more particularly described in the following description and examples that are intended to be illustrative only since numerous modifications and variations therein will be apparent to those skilled in the art. As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of”

The present invention relates to a chewable electrolyte tablet. As used herein, a “chewable” electrolyte tablet is one that is palatable and may be chewed and ingested with little or no water. As used herein, “little or no water” refers to less than about 1 fluid ounce of water or any other consumable liquid. As such, in one aspect, the present invention provides an electrolyte tablet that may be ingested by a person or animal through simple mastication of the tablet and without the need for any water to dissolve the tablet, unlike prior art electrolyte powders. Accordingly, the person ingesting the tablet may decide the amount of water, if any, that they wish to take, thereby reducing the risk of consuming too much water or any other consumable liquid which, as previously discussed, could have deleterious side effects.

One of the reasons prior art tablets and powders require water is to dilute the electrolyte mixture. Dilution is generally necessary as the concentrated doses of the vitamins, minerals and/or amino acids in prior art tablets and powders may cause gastrointestinal problems to the user of the tablet or powder due to the higher levels of salts used to deliver one or more of these vitamins, minerals and/or amino acids. The present invention overcomes this requirement for water through the use of a gastrointestinal soothing agent. As used herein, a “gastrointestinal soothing agent” is any composition added to the tablet to prevent stomach irritation that may be caused by the salts in the tablet, thereby achieving better tolerance without the need for water or any other consumable liquid. In one embodiment, Aloe Vera is used as the gastrointestinal soothing agent. The extract or leaf powder may be used. In an alternative embodiment, bismuth subsalicylate is used as the gastrointestinal soothing agent. Other gastrointestinal soothing agents that may be used in the present invention include, but are not limited to, calcium carbonate, magnesium hydroxide, famotidine, cimetidine HCl, ranitidine HCl, esomeprazole magnesium, or a combination thereof.

The amount of the gastrointestinal soothing agent used in the present invention may vary depending on the other ingredients used in the chewable electrolyte tablet. In general, the greater the concentration of salts and/or other possible stomach irritants, the greater the amount of the gastrointestinal soothing agent that is used. In one embodiment, the gastrointestinal soothing agent is present in an amount from about 0.1 to about 10% by weight of the composition. In another embodiment, the gastrointestinal soothing agent is present in an amount from about 1 to about 5% by weight of the composition.

In another aspect, the present invention provides a chewable electrolyte tablet having an improved taste, unlike prior art electrolyte tablets and powders. Due to the improved taste, the tablets of the present invention are actually chewable and digestible without the need for water or any other consumable liquid. As prior art tablets and powders have poor taste, they can not be chewed and, in fact, are not designed to be chewed. Rather, these tablets and powders require water or another fluid to be used to swallow the tablet or powder. As the chewable tablets of the present invention are palatable, they may be chewed and digested without water, again providing the advantage of permitting the person ingesting the chewable tablet to decide the amount of water or any other consumable liquid, if any, which they wish to take with the tablet.

The chewable electrolyte tablets of the present invention are, in one embodiment, palatable even though the tablets of the present invention are substantially carbohydrate-free. As used herein, “substantially carbohydrate-free” is meant to include any tablet having little, if any, sugars or other carbohydrates. As a result, these tablets do not increase the caloric intake of the person ingesting the tablet and, therefore, enhance the calorie burning aspects of any physical activity, unlike prior art electrolyte drinks and powders that include substantial levels of sugars or other carbohydrates. In one embodiment, the amount of sugars or other carbohydrates is less than about 5% by weight of the overall composition. In another embodiment, the amount of sugars or other carbohydrates is less than about 3% by weight of the overall composition. In yet another embodiment, the amount of sugars or other carbohydrates is less than about 2% by weight of the overall composition.

The chewable electrolyte tablets of the present invention are palatable even though they are substantially carbohydrate-free through the use, in one embodiment, of a non-carbohydrate sweetening agent. As used herein, a “non-carbohydrate sweetening agent” is intended to include a sugar alcohol, an artificial sweetener, or both. By using a sugar alcohol or artificial sweetener, the chewable electrolyte tablets of the present invention have a pleasant sweetened taste without the addition of sugars or other carbohydrates. The amount of sugar alcohol, artificial sweetener, or both used in the present invention may range, in one embodiment, from about 1 to about 90% by weight of the overall composition. In another embodiment, the amount of sugar alcohol, artificial sweetener, or both used in the present invention may range, in another embodiment, from about 30 to about 60% by weight of the overall composition.

Any sugar alcohol or artificial sweetener may be used in the present invention that may provide good taste as well as physical function in the tablet. Examples of artificial sweeteners that may be used in the present invention include, but are not limited to, sucralose, aspartame, saccharine, acesulfame potassium, or a combination thereof. Example of sugar alcohols that may be used include, but are not limited to, dannitol, xylitol, lacitol, isomalt, mannitol, erythritol, maltitol, sorbitol, fatty acid esters, or a combination thereof.

In addition to or as an alternative to the sugar alcohols and/or artificial sweeteners, the chewable electrolyte tablets of the present invention may be made more palatable through the use of a flavoring agent. The flavoring agent that may be used is any agent that may provide a pleasant and/or more palatable taste to the chewable electrolyte tablet. Examples of flavoring agents that may be used include, but are not limited to, citric acid, one or more fruit flavors, such as lemon, cherry, lime, grape, orange, or the like, cocoa powder, calcium citrate, black tea, green tea, ginseng, ginkgo, chamomile, peppermint, fennel, ginger, licorice, lotus seed, sarsaparilla, nutmeg, cinnamon, jasmine, sugar cane, vanilla, coffee, or a combination thereof.

The amount of flavoring agent that may be used in the present invention may range, in one embodiment, from about 0 to about 20% by weight of the overall composition. In another embodiment, the amount of flavoring agent that may be used in the present invention may range, in another embodiment, from about 2 to about 5% by weight of the overall composition.

The compositions of the present invention may also utilize other agents to make the compositions more palatable and, therefore, chewable. In one embodiment, the chewable electrolyte tablets of the present invention may also utilize encapsulated salts based on fatty acids, cellulose and/or synthetic polymer coating materials to enhance flavor and improve mouth feel.

The chewable electrolyte tablets of the present invention are used to deliver nutritional value to the person or animal ingesting the tablet. As a result, the tablets include one or more nutritional compounds selected from electrolytes, vitamins, and/or amino acids, depending on the selected formulation and the intended benefits to be delivered to the person or animal ingesting the tablet. Accordingly, it one embodiment, the chewable electrolyte tablets of the present invention include at least one electrolyte. Examples of electrolytes that may be used in the present invention include, but are not limited to, magnesium, zinc, chromium, sodium, chloride, potassium, calcium, or a combination thereof.

In an alternative embodiment, the chewable electrolyte tablets of the present invention may include at least one amino acid. Amino acids useful in the present invention include, but are not limited to, one or more branched amino acids, or amino acids in general, such as alanine, arginine, asparagine, aspartic acid, cysteine, cystine, glutamine, glutamic acid, glycine, histidine, hydroxyproline, isoleucine, leucine, lysine, methionine, omithine, phenylalanine, proline, serine, threonine, tryptophan, tyrosine, valine, or a combination thereof. The amount of amino acids included in the present invention may vary from about 0 to about 25% by weight of the composition. In an alternative embodiment, the amount of amino acids included in the tablet may range from about 5 to about 25% by weight of the composition.

In yet another alternative embodiment, the chewable electrolyte tablets of the present invention may include at least one vitamin. Vitamins useful in the present invention include, but are not limited to, niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iodine, ascorbic acid, pyridoxine, or a combination thereof. The amount of vitamins included in the present invention may vary from about 0 to about 20% by weight of the composition. In an alternative embodiment, the amount of vitamins included in the tablet may range from about 3 to about 12% by weight of the composition.

In one aspect of the present invention, the chewable electrolyte tablets may include potassium ion. Potassium ion may be utilized to combat stress and sugar absorption in the gastro intestinal tract. The salt form of potassium may be used in the present invention, such as potassium chloride, potassium citrate, potassium maleate, or a combination thereof, as well as any inorganic or organic potassium salt. The amount of potassium included in the present invention may vary from about 0 to about 10% by weight of the composition. In an alternative embodiment, the amount of potassium included in the tablet may range from about 1 to about 5% by weight of the composition.

In another aspect of the present invention, the chewable electrolyte tablets may include zinc. Zinc is an electrolyte involved in energy generation. The salt form of zinc may be used in the present invention, such as zinc oxide, zinc sulfate, zinc glycinate, or a combination thereof, as well as any organic or inorganic zinc salt. The amount of zinc included in the present invention may vary from about 0 to about 5% by weight of the composition. In an alternative embodiment, the amount of zinc included in the tablet may range from about 0.5 to about 3% by weight of the composition.

In another aspect of the present invention, the chewable electrolyte tablets may include sodium. Sodium ion is utilized for fluid balance. The use of sodium may be minimized since it is not as required as other minerals. The salt form of sodium may be used in the present invention, such as sodium chloride, sodium bicarbonate, sodium citrate, or any other organic or inorganic sodium salt. The amount of sodium included in the present invention may vary from about 0 to about 10% by weight of the composition. In an alternative embodiment, the amount of sodium included in the tablet may range from about 1 to about 5% by weight of the composition.

In yet another aspect of the present invention, the chewable electrolyte tablets may include chloride. Chloride is an essential electrolyte involved in fluid balance and for muscle tone. The salt form of chloride may be used in the present invention, such as sodium chloride, potassium chloride, or any other organic or inorganic chloride salt. The amount of chloride included in the present invention may vary from about 0 to about 10% by weight of the composition. In an alternative embodiment, the amount of chloride included in the tablet may range from about 1 to about 5% by weight of the composition.

In still another aspect of the present invention, the chewable electrolyte tablets may include magnesium. Magnesium is essential for use in muscle tone. When magnesium concentrations are low in the blood stream cramping may occur. The salt form of magnesium may be used in the present invention, such as magnesium oxide, magnesium chloride, magnesium sulfate, magnesium glycinate, or any other organic or inorganic magnesium salt. The amount of magnesium included in the present invention may vary from about 0 to about 10% by weight of the composition. In an alternative embodiment, the amount of magnesium included in the tablet may range from about 2 to about 7% by weight of the composition.

In yet another aspect of the present invention, the chewable electrolyte tablets may include chromium. Chromium is utilized to enhance the efficiency of energy conversion and to help in weight control. The salt form of chromium may be used in the present invention, such as chromium chloride, chromium gycinate, chromium picolinate, or any other organic or inorganic chromium salt. The amount of chromium included in the present invention may vary from about 0 to about 3% by weight of the composition. In an alternative embodiment, the amount of chromium included in the tablet may range from about 0.01 to about 1% by weight of the composition.

In still another aspect of the present invention, the chewable electrolyte tablets may include pyridoxine in combination with ascorbic acid. Pyridoxine in combination with ascorbic acid greatly reduces the stress effect on the metabolic system of the body by helping increase kidney function for blood purification. The form of these compounds may be pyridoxine HCl, pyrophosphate, or any other salt form. Ascorbic acid may be ascorbic acid, palmitate, sodium ascorbate, or any other salt form. The amount of pyridoxine and/or ascorbic acid included in the present invention may vary from about 0 to about 15% by weight of the composition. In an alternative embodiment, the amount of pyridoxine and/or ascorbic acid included in the tablet may range from about 2 to about 7% by weight of the composition.

In yet another aspect of the present invention, the chewable electrolyte tablets may include calcium. Calcium is a key active for muscle spasms and also osmolarity of blood and cells. The salt form of calcium may be used in the present invention, such as calcium carbonate, calcium citrate, calcium maleate, calcium phosphate or any other inorganic or organic calcium salt. The amount of calcium included in the present invention may vary from about 0 to about 15% by weight of the composition. In an alternative embodiment, the amount of calcium included in the tablet may range from about 2 to about 10% by weight of the composition.

In another embodiment of the present invention, the chewable electrolyte tablets are designed to release the electrolytes, vitamins, and/or amino acids in a sustained-release or time-release manner. As used herein, “time-release” and “sustained-release” are used interchangeably to define a composition that slowly releases the nutrients contained within the composition over a period of time. This is especially beneficial in specific situations such as the military activities or athletes in motion (such as bicycling, running). In a first embodiment, the nutrients are released over a period of from about 2 hours to about 24 hours. In another embodiment, the nutrients are released over a period of time of from about 4 hours to about 24 hours. In yet another embodiment, the nutrients are released over a period of time of from about 4 hours to about 12 hours. In still another embodiment, the nutrients are released over a period of time greater than about 8 hours.

It is to be understood that while the invention has been described in conjunction with the specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

### EXAMPLES

### Example 1

In Example 1, an electrolyte composition having the following formulation: The delivery system may be devised as follows:

| INGREDIENT | DOSE RANGE POSSIBLE |
|---|---|
| Magnesium (oxide) | (5 mg to 200 mg) |
| Zinc (Citrate) (mircroencapsualted) | (1 mg to 40 mg) |
| Chromium (polynicotinate) | (0.005 mg to 0.200 mg) |
| Sodium (Chloride)(microencapsulated) | (1 mg to 700 mg) |
| Chloride (Sodium)(microencapsulated) | (1 mg to 700 mg) |
| Potassium (Chloride)(microencapsulated) | (1 mg to 700 mg) |
| Calcium (Carbonate) | (1 mg to 1000 mg) |
| Ascorbic acid | (1 mg to 700 mg) |
| Pyridoxine (HCl) | (0.1 mg to 100 mg) |
| Xylitol | (100 mg to 3000 mg) |
| Mannitol | (100 mg to 3000 mg) |
| Citric Acid | (1 mg to 400 mg) |
| Lemon Flavor | (1 mg to 300 mg) |
| Lime Flavor | (1 mg to 300 mg) |
| Aloe Vera | (1 mg to 300 mg) |

It is to be understood that while the chewable electrolyte compositions of the present invention are substantially carbohydrate-free, it may be beneficial, in some alternative embodiments to include some levels of carbohydrates. These embodiments are generally those that would be used by an individual for whom the excess calories would not be problematic, such as those embodiments wherein the chewable tablets are used as fast energy for athletes or patients under stress conditions. Carbohydrates that may be used in the present invention include, but are not limited to, monosaccharides, disaccharides, polysaccharides, and oligiosaccharides. These may include dextrose, fructose, lactose, glucose, sucrose, cellulose derivatives, such as microcrystalline cellulose (MCC), starch, or any other form of saccharide. The amounts of carbohydrates may be from about 10 to about 50% by weight of the composition.

Although the illustrative embodiments of the present disclosure have been described herein with reference to the accompanying drawings and examples, it is to be understood that the disclosure is not limited to those precise embodiments, and various other changes and modifications may be affected therein by one skilled in the art without departing from the scope of spirit of the disclosure. All such changes and modifications are intended to be included within the scope of the disclosure as defined by the appended claims.

We claim:

1. An electrolyte tablet comprising:
at least one nutritional compound; and
a non-carbohydrate sweetening agent;
wherein the electrolyte tablet is chewable such that it may be ingested with little or no water.

2. The electrolyte tablet of claim 1, wherein the at least one nutritional compound is selected from at least one electrolyte, at least one vitamin, at least one amino acid, or a combination thereof.

3. The electrolyte tablet of claim 2, wherein the at least one electrolyte is selected from magnesium, zinc, chromium, sodium, chloride, potassium, calcium, or a combination thereof.

4. The electrolyte tablet of claim 2, wherein the at least one amino acid is selected from alanine, arginine, asparagine, aspartic acid, cysteine, cystine, glutamine, glutamic acid, glycine, histidine, hydroxyproline, isoleucine, leucine, lysine, methionine, omithine, phenylalanine, proline, serine, threonine, tryptophan, tyrosine, valine, or a combination thereof.

5. The electrolyte tablet of claim 2, wherein the at least one amino acid is present in an amount from about 0 to about 25% by weight of the electrolyte tablet.

6. The electrolyte tablet of claim 2, wherein the at least one vitamin is selected from niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iodine, ascorbic acid, pyridoxine, or a combination thereof.

7. The electrolyte tablet of claim 2, wherein the at least one vitamin is present in an amount from about 0 to about 20% by weight of the electrolyte tablet.

8. The electrolyte tablet of claim 1, further comprising a gastrointestinal soothing agent.

9. The electrolyte tablet of claim 8, wherein the gastrointestinal soothing agent is selected from Aloe Vera, bismuth subsalicylate, calcium carbonate, magnesium hydroxide, famotidine, cimetidine HCl, ranitidine HCl, esomeprazole magnesium, or a combination thereof.

10. The electrolyte tablet of claim 8, wherein the gastrointestinal soothing agent is present in an amount of from about 0.1 to about 10% by weight of the electrolyte tablet.

11. The electrolyte tablet of claim 1, wherein the non-carbohydrate sweetening agent is selected from dannitol, xylitol, lacitol, isomalt, mannitol, acesulfame potassium, erythritol, maltitol, sorbitol, fatty acid esters, sucralose, aspartame, saccharine, or a combination thereof.

12. The electrolyte tablet of claim 11, wherein the non-carbohydrate sweetening agent is present in an amount of from about 1 to about 90% by weight of the electrolyte tablet.

13. The electrolyte tablet of claim 1, further comprising a flavoring agent.

14. The electrolyte tablet of claim 13, wherein the flavoring agent is selected from citric acid, lemon flavor, cherry flavor, lime flavor, grape flavor, orange flavor, cocoa powder, calcium citrate, black tea, green tea, ginseng, ginkgo, chamomile, peppermint, fennel, ginger, licorice, lotus seed, sarsaparilla, nutmeg, cinnamon, jasmine, sugar cane, vanilla, coffee, or a combination thereof.

15. The electrolyte tablet of claim 13, wherein the flavoring agent is present in an amount of from about 0 to about 20% by weight of the electrolyte tablet.

16. A sustained-release electrolyte tablet comprising:
at least one sustained-release nutritional compound;
wherein the at least one sustained-release nutritional compound is released over a period of time greater than about 2 hours.

17. The electrolyte tablet of claim 16, wherein the at least one sustained-release nutritional compound is selected from at least one sustained-release electrolyte, at least one sustained-release vitamin, at least one sustained-release amino acid, or a combination thereof.

18. The electrolyte tablet of claim 17, wherein the at least one sustained-release electrolyte is selected from magnesium, zinc, chromium, sodium, chloride, potassium, calcium, or a combination thereof.

19. The electrolyte tablet of claim 17, wherein the at least one sustained-release amino acid is selected from alanine, arginine, asparagine, aspartic acid, cysteine, cystine, glutamine, glutamic acid, glycine, histidine, hydroxyproline, isoleucine, leucine, lysine, methionine, omithine, phenylalanine, proline, serine, threonine, tryptophan, tyrosine, valine, or a combination thereof.

20. The electrolyte tablet of claim 19, wherein the at least one sustained-release amino acid is present in an amount from about 0 to about 25% by weight of the electrolyte tablet.

21. The electrolyte tablet of claim 17, wherein the at least one sustained-release vitamin is selected from niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iodine, ascorbic acid, pyridoxine, or a combination thereof.

22. The electrolyte tablet of claim 21, wherein the at least one sustained-release vitamin is present in an amount from about 0 to about 20% by weight of the electrolyte tablet.

23. The electrolyte tablet of claim 16, further comprising a gastrointestinal soothing agent.

24. The electrolyte tablet of claim 23, wherein the gastrointestinal soothing agent is selected from Aloe Vera, bismuth subsalicylate, calcium carbonate, magnesium hydroxide, famotidine, cimetidine HCl, ranitidine HCl, esomeprazole magnesium, or a combination thereof.

25. The electrolyte tablet of claim 23, wherein the gastrointestinal soothing agent is present in an amount of from about 0.1 to about 10% by weight of the electrolyte tablet.

26. The electrolyte tablet of claim 16, further comprising a non-carbohydrate sweetening agent.

27. The electrolyte tablet of claim 26, wherein the non-carbohydrate sweetening agent is selected from dannitol, xylitol, lacitol, isomalt, mannitol, acesulfame potassium, erythritol, maltitol, sorbitol, fatty acid esters, sucralose, aspartame, saccharine, or a combination thereof.

28. The electrolyte tablet of claim 26, wherein the non-carbohydrate sweetening agent is present in an amount of from about 1 to about 90% by weight of the electrolyte tablet.

29. The electrolyte tablet of claim 16, further comprising a flavoring agent.

30. The electrolyte tablet of claim 29, wherein the flavoring agent is selected from citric acid, lemon flavor, cherry flavor, lime flavor, grape flavor, orange flavor, cocoa powder, calcium citrate, black tea, green tea, ginseng, ginkgo, chamomile, peppermint, fennel, ginger, licorice, lotus seed, sarsaparilla, nutmeg, cinnamon, jasmine, sugar cane, vanilla, coffee, or a combination thereof.

31. The electrolyte tablet of claim 29, wherein the flavoring agent is present in an amount of from about 0 to about 20% by weight of the electrolyte tablet.

32. The electrolyte tablet of claim 16, wherein the electrolyte tablet is chewable such that it may be ingested with little or no water.

33. A chewable electrolyte tablet comprising:
a composition having the following formulation:

| INGREDIENT | DOSE RANGE POSSIBLE |
|---|---|
| Magnesium | (5 mg to 200 mg) |
| Zinc | (1 mg to 40 mg) |
| Chromium | (.005 to .200 mg) |
| Sodium | (1 mg to 700 mg) |
| Chloride | (1 mg to 700 mg) |
| Potassium | (1 mg to 700 mg) |
| Calcium | (1 mg to 1000 mg) |
| Ascorbic acid | (1 mg to 700 mg) |
| Pyridoxine (HCl) | (.1 mg to 100 mg) |
| Non-Carbohydrate sweetening agent | (100 mg to 3000 mg) |
| Flavoring agent | (1 mg to 400 mg) |
| Gastrointestinal soothing agent | (1 mg to 300 mg) |

wherein the electrolyte tablet is chewable such that it may be ingested with little or no water.

34. The electrolyte tablet of claim 33, further comprising at least one amino acid selected from alanine, arginine, asparagine, aspartic acid, cysteine, cystine, glutamine, glutamic acid, glycine, histidine, hydroxyproline, isoleucine, leucine, lysine, methionine, omithine, phenylalanine, proline, serine, threonine, tryptophan, tyrosine, valine, or a combination thereof.

35. The electrolyte tablet of claim 34, wherein the at least one amino acid is present in an amount from about 0 to about 25% by weight of the electrolyte tablet.

36. The electrolyte tablet of claim 33, further comprising at least one vitamin selected from niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iodine, ascorbic acid, pyridoxine, or a combination thereof.

37. The electrolyte tablet of claim 36, wherein the at least one vitamin is present in an amount from about 0 to about 20% by weight of the electrolyte tablet.

38. The electrolyte tablet of claim 33, wherein the gastrointestinal soothing agent is selected from Aloe Vera, bismuth subsalicylate, calcium carbonate, magnesium hydroxide, famotidine, cimetidine HCl, ranitidine HCl, esomeprazole magnesium, or a combination thereof.

39. The electrolyte tablet of claim 33, wherein the non-carbohydrate sweetening agent is selected from dannitol, xylitol, lacitol, isomalt, mannitol, acesulfame potassium, erythritol, maltitol, sorbitol, fatty acid esters, sucralose, aspartame, saccharine, or a combination thereof.

40. The electrolyte tablet of claim 33, wherein the flavoring agent is selected from citric acid, lemon flavor, cherry flavor, lime flavor, grape flavor, orange flavor, cocoa powder, calcium citrate, black tea, green tea, ginseng, ginkgo, chamomile, peppermint, fennel, ginger, licorice, lotus seed, sarsaparilla, nutmeg, cinnamon, jasmine, sugar cane, vanilla, coffee, or a combination thereof.
